(19) United States
(12) Patent Application Publication
(10) Pub. No.: US 2026/0060707 A1
(43) Pub. Date: Mar. 5, 2026

(54) SHOCK WAVE CATHETER WITH EMITTER CENTERING

(71) Applicant: Shockwave Medical, Inc., Santa Clara, CA (US)

(72) Inventors: Rainier BETELIA, San Jose, CA (US); Todd Weston JENKINS, Mountain View, CA (US); Nancy P. NGUYEN, San Jose, CA (US); Danielle Sheree ROND, Santa Clara, CA (US)

(73) Assignee: Shockwave Medical, Inc., Santa Clara, CA (US)

(21) Appl. No.: 19/310,395

(22) Filed: Aug. 26, 2025

Related U.S. Application Data
(60) Provisional application No. 63/688,219, filed on Aug. 28, 2024.

(51) Int. Cl. A61B 17/22; A61M 25/00; A61M 25/01

(52) U.S. Cl. CPC A61B 17/22022; A61M 25/0026; A61M 25/0108; A61B 2017/22025

(57) ABSTRACT

A catheter includes one or more shock wave generating or emitting sources mounted on or embedded in a main body lumen in positioned within an enclosure, where the enclosure has multiple regions or segments, and wherein one or more of the shock wave sources are located in one or more of the enclosure segments. In some aspects, the enclosure includes a narrowing between regions, in part defining the plurality of segments, the narrowing further functioning to keep the main body lumen centered along the length of the enclosure, thereby maintaining the shock wave sources an operational distance away from the material of the enclosure.

### CROSS-REFERENCE TO RELATED APPLICATIONS

This application claims priority to and the benefit of U.S. Provisional Application No. 63/688,219, filed Aug. 28, 2024, the contents of which are incorporated herein in its entirety.

### FIELD OF THE DISCLOSURE

The present disclosure relates generally to the field of medical devices and therapeutic methods, and more specifically to acoustic energy generating assemblies for inclusion in shock wave catheter devices used for treating lesions in a body lumens and tissues, such as calcified lesions and occlusions in vasculature, calcific heart valve stenosis, or kidney stones in the urinary system.

### BACKGROUND

Stenosis in body lumens can negatively impact patient health. For example, when vascular plaque builds up in the walls of the coronary arteries, the accumulation can restrict blood flow to the heart muscle, which can eventually lead to a heart attack. Treating the stenosis is even more challenging when the plaque becomes calcified. Catheter devices are one type of device that can be used to treat calcified lesions in a body lumen. When treating lesions with a catheter device, it is important to minimize damage to surrounding soft tissues while still breaking up the lesion as much as possible.

A wide variety of catheters have been developed for treating stenotic blood vessels that are narrowed by the progressive growth and accumulation of plaque, a condition also known as atherosclerosis. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature. A particular focus is to treat calcified lesions of plaque, such as calcified lesions in the vasculature associated with arterial disease.

However, traditional dilation balloon angioplasty therapies may not work with calcified tissue because the calcium in the atherosclerotic plaque hardens the lesion, resisting the mechanical force of balloon expansion. The resistance can result in more procedural complication and vessel damage because the high-pressure balloons preferentially expand away from the hard calcified tissue. The predisposition of the ballon to expand in a direction of lower resistance increases the risk of major dissection or perforation of the vessel, often at the ends of a lesion at the interface between healthy tissue and calcified tissue (i.e., where the balloon encounters soft tissue). In the case of an eccentric calcified lesion where the hardened region is biased on a side of a vessel, the expansion ends up going preferentially in the direction opposite of the calcified region of the lesion, straining and dissecting the healthier side of the blood vessel. Moreover, in the case of nodular calcium, expansion of a standard angioplasty balloon can lead to pushing the node of calcified material in a manner that may puncture the vessel.

Another approach to dealing with calcified stenotic plaque is to cut away at a calcified lesion, by using a cutting or scoring balloon, an angioplasty balloon having a raised structure on the surface of the balloon (e.g., an angioplasty balloon with blade-like structures on its exterior). The expansion of an angioplasty balloon having a raised structure may allow for mechanical force on a lesion to be focused at the location of the raised structure, but these devices still do not provide for any protection from dissection or perforation resulting from preferential expansion of the balloon away from hardened tissue. Another technique for cutting away at a calcified lesion is by using an atherectomy device, which typically includes a motor-driven rotating or oscillating blade that is pushed into and cuts through an occlusion (also referred to as “debulking” or “extirpation”). Because these treatments work by liberating the calcified tissues from a blood vessel wall, there is an increased risk of embolism where the free-floating masses of calcification may proceed down the blood stream. Such systems may include baskets to capture or negative-pressure lumens to aspirate such unmoored emboli as a necessary additional structure to ensure the safety of such devices. An additional concern for atherectomy devices is that the movement or rotation of atherectomy catheter blades generates frictional heat and can cause a related thermal injury from mere operation of the atherectomy device. That heat can directly injure the lining of a blood vessel and can also lead to an increased risk of blood clotting. Naturally, the action of a moving blade within the vasculature also significantly increases the potential for a large dissection and perforation of the blood vessel by the blade itself.

Accordingly, there is an ongoing need for medical devices that can address and treat calcified plaques and stenosis with a safety profile that minimizes risk of blood vessel damage and with an efficacy profile that provides for durable circulatory restoration

### SUMMARY

According to aspects of the disclosure, a shock wave catheter includes an emitter centering feature that keeps an inner shaft of the catheter centered (e.g., concentric) with an enclosure of the catheter. In some examples, the inner shaft is only in contact with (e.g., sealed to) proximal and distal ends of the enclosure. The emitter centering feature can be a narrowed region of the enclosure. Shock wave emitters of the catheter may be enclosed by the enclosure and located on both sides of the narrowed region. The narrowed region (otherwise referred to herein as a narrowing) may fluidly connect the enclosure segments on either side of the narrowing. In some examples, the shock wave catheter includes an enclosure having more than one narrowed region, such as two narrowed regions. In such implementations, the enclosure can be referred to as having multiple regions, or segments, having a serial or sequential arrangement.

According to aspects of the disclosure, a shock wave catheter includes a plurality of enclosures that are aligned along an inner shaft of the catheter. Shock wave emitters may be enclosed with each of the plurality of enclosures. Each of the plurality of enclosures can be sealed to the inner shaft to keep the inner shaft centered relative to the plurality of enclosures.

Shock wave catheters having an extended distribution of shock wave emitters can be advantageous in that they may require the catheter to be moved less frequently, or not at all, to treat longer lesions. However, in treating longer lesions in tortuous vasculature, long and flexible enclosures surrounding the emitters may have the tendency to offset relative to the inner shaft when the catheter is bent. This offset increases the risk of the emitters contacting the enclosure, which could cause the enclosure to rupture upon shock wave generation at the emitters. Additionally, or alternatively, the shock wave treatment applied to the lesion could exceed the intended parameters by being too close to the enclosure.

Using the shock wave catheters having narrowed region(s) (or multiple enclosures in other examples) disclosed herein, the emitters can remain centered relative to the enclosure, in particular when using the catheter to treat long lesions in tortuous vasculature. The centering provided from the shock wave catheters disclosed herein may ensure a safe operating distance exists between the shock wave emitters and the enclosure, thus preserving the integrity of the enclosure and adhering to the prescribed operating conditions of the system. Moreover, the centering improved by the narrowed regions or multiple enclosures can increase the possible longitudinal distribution of shock wave emitters in a catheter, thus enabling the treatment of longer lesions without requiring repositioning of the catheter during use.

In some aspects, a shock wave catheter is provided, including: an enclosure including a distal segment, a proximal segment, and a narrowing region fluidly connecting the distal segment and the proximal segment; an inner shaft extending through the distal segment and the proximal segment; a distal shock wave emitter supported by the inner shaft and located within the distal segment; and a proximal shock wave emitter mounted on the inner shaft and located within the proximal segment.

In some aspects, the distal emitter includes a plurality of distal emitters and the proximal emitter includes a plurality of proximal emitters. In some aspects, the enclosure includes a middle segment located between the distal segment and the proximal segment and a plurality of narrowing regions separating each of the distal segment, the proximal segment, and the middle segment, and wherein the catheter further includes a middle shock wave emitter mounted on the inner shaft and located within the middle segment. In some aspects, the enclosure is configured such that spacing is maintained between the inner shaft and the distal and the proximal segments at a maximum curvature of the catheter. In some aspects, at least one of the distal segment and the proximal segment has a working length of no more than 5 mm. In some aspects, the narrowing has a narrower width than the distal segment and the proximal segment when the enclosure is filled and pressurized to between two and four atmospheres. In some aspects, a width of the narrowing is less than 50% of a width of at least one of the distal segment and the proximal segment. In some aspects, a length of the narrowing is less than a distance between the distal and proximal shock wave emitters. In some aspects, the catheter includes a radiopaque marker disposed at the narrowing. In some aspects, the catheter includes a fluid lumen configured to fill the proximal segment and the distal segment with conductive fluid and to remove gas from the proximal segment and the distal segment of the enclosure. In some aspects, the fluid lumen includes a single opening disposed at a distal portion of the shock wave catheter. In some aspects, the fluid lumen includes a first opening in the proximal segment and a second opening in the distal segment of the enclosure. In some aspects, the catheter includes a first fluid lumen configured to fill the proximal segment and the distal segment of the enclosure with conductive fluid, and a second fluid lumen configured to remove gas from the proximal segment and the distal segment of the enclosure.

In some aspects, a system is provided, including: the shock wave catheter of any of the above aspects; and a pulse generator electrically coupled to the proximal shock wave emitter and the distal shock wave emitter, the pulse generator configured to generate voltage pulses that cause shock wave formation at the proximal shock wave emitter and the distal shock wave emitter.

In some aspects, a method for treating a lesion in a body lumen is provided, including: advancing a shock wave catheter within the body lumen such that an enclosure of the shock wave catheter is positioned proximate to the lesion, the enclosure of the catheter including a proximal segment, a distal segment, and a narrowing region fluidly connecting the proximal segment and the distal segment; and generating shock waves by a proximal shock wave emitter of the shock wave catheter that is located within the proximal segment and a distal shock wave emitter of the shock wave catheter that is located within the distal segment to treat the lesion.

In some aspects, advancing the shock wave catheter within the body lumen includes bending the shock wave catheter such that the proximal and distal segment are proximate to the lesion, and wherein, when the shock wave catheter is bent, the proximal and distal shock wave emitters are spaced from the respective proximal and distal segments due to the narrowing region.

In some aspects, a shock wave catheter is provided, including: a distal enclosure; a proximal enclosure positioned proximally of the distal enclosure; an inner shaft extending through the distal enclosure and the proximal enclosure; a distal shock wave emitter supported by the inner shaft and enclosed by the distal enclosure; and a proximal shock wave emitter supported by the inner shaft and enclosed by the proximal enclosure.

In some aspects, the catheter includes a first fluid lumen in fluid communication with the distal enclosure and a second fluid lumen in fluid communication with the proximal enclosure. In some aspects, the distal enclosure and the proximal enclosure are configured to be filled separately. In some aspects, the catheter includes a fluid lumen fluidly connected to the distal enclosure and the proximal enclosure, the fluid lumen including a first opening fluidly connected to the distal enclosure and a second opening fluidly connected to the proximal enclosure.

### DESCRIPTION OF THE FIGURES

Illustrative aspects of the present disclosure are described in detail below with reference to the following figures. It is intended that the embodiments and figures disclosed herein are to be considered illustrative and exemplary rather than restrictive.

FIG. 1 illustrates an exemplary shock wave catheter system, according to aspects of the disclosure.

FIGS. 2A-2C illustrate an exemplary shock wave catheter having an enclosure with a narrowed region that can be used in the system of FIG. 1, according to aspects of the disclosure; FIG. 2A illustrates a side view of the catheter, according to aspects of the disclosure; FIG. 2B illustrates an enlarged view of the narrowed region of the catheter, according to aspects of the disclosure; and FIG. 2C illustrates a perspective view of the catheter, according to aspects of the disclosure.

FIGS. 3A-3B illustrate an exemplary shock wave catheter having an enclosure with more than one narrowed region that can be used in the system of FIG. 1, according to aspects of the disclosure; FIG. 3A illustrates a side view of the catheter, according to aspects of the disclosure; and FIG. 3B illustrates a perspective view of the catheter, according to aspects of the disclosure.

FIGS. 4A-4C illustrate diagrams of exemplary shock wave catheters including fluidly connected enclosure segments that can be used in the system of FIG. 1, according to aspects of the disclosure; FIG. 4A illustrates a catheter including separate fluid lumens for filling and purging the segments of the enclosure, according to aspects of the disclosure; FIG. 4B illustrates a catheter including a fluid lumen for both filling and purging the enclosure, according to aspects of the disclosure; and FIG. 4C illustrates a catheter including a fluid lumen having a single opening at the distal portion of the catheter for both filling and purging the enclosure, according to aspects of the disclosure.

FIGS. 5A-5B illustrate diagrams of exemplary shock wave catheters including multiple enclosures that can be used in the system of FIG. 1, according to aspects of the disclosure; FIG. 5A illustrates a catheter including a single fluid lumen for filling and/or purging each of the enclosures, according to aspects of the disclosure; and FIG. 5B illustrates a catheter including multiple fluid lumens for respectively filling and/or purging the enclosures, according to aspects of the disclosure.

FIGS. 6A-6B illustrate narrowed regions of exemplary shock wave catheters having a radiopaque marker that can be used in the system of FIG. 1, according to aspects of the disclosure; FIG. 6A illustrates a narrowed region having a radiopaque marker on the enclosure of the shock wave catheter, according to aspects of the disclosure; and FIG. 6B illustrates a narrowed region having a radiopaque marker on the inner shaft of the shock wave catheter, according to aspects of the disclosure.

FIG. 7 illustrates an exemplary shock wave catheter including an enclosure with narrowed regions that can be used in the system of FIG. 1, the shock wave catheter traversing tortuous vasculature, according to aspects of the disclosure.

FIG. 8 illustrates an exemplary computing system configured to be used with the shock wave catheter of FIG. 1, according to aspects of the disclosure.

### DETAILED DESCRIPTION

The following description is presented to enable a person of ordinary skill in the art to make and use the various embodiments and aspects thereof disclosed herein. Descriptions of specific catheters, systems, methods, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

In recent years, in order to treat atherosclerosis and related conditions, the technique and treatment of intravascular lithotripsy (“IVL”) has been developed, which is an interventional procedure to modify calcified plaque in diseased vasculature. More precisely, IVL is the energy-based generation of ultrasonic acoustic pressure waves for modification, fracture, and fragmentation of vascular calcification in situ. The mechanism of plaque modification is through use of a catheter having one or more ultrasonic short pressure pulses (commonly referred to as “shock waves”) emit from a generating source located within a liquid that can create acoustic ultrasonic shock waves that modify and fragment the calcified plaque. IVL devices vary in design with respect to the energy source used to generate the acoustic shock waves, with two exemplary energy sources being electrohydraulic generation and laser generation.

For electrohydraulic generation of ultrasonic short pressure pulses, a conductive solution (e.g., saline) can be contained within an enclosure that surrounds electrodes or can be flushed through a tube that surrounds the electrodes. The calcified plaque modification is achieved by creating ultrasonic shock waves within the catheter by an electrical discharge (e.g., a plasma arc) across the electrodes. The energy from this electrical discharge enters the surrounding fluid, generating an acoustic shock wave where the wave itself is ultrasonic (i.e., a wave that has frequency components of greater than 20,000 Hz). In addition, the discharge creates one or more rapidly expanding and collapsing vapor bubbles that generate secondary shock waves due to the cavitation of the collapsing vapor bubble. The shock waves propagate radially outward and modify calcified plaque within the blood vessels. The shock waves travel deeply and safely through soft arterial tissue because of the acoustic impedance soft tissue, which is similar to water. Acoustic impedance is a function of the density and the elasticity of a material and the speed of sound through that material. When the shock waves encounter tissues with a different acoustic impedance, such as intimal calcification of plaque close to the surface or endothelium of a vessel or medical calcification in the smooth muscle layer of a vessel, the leading edge of the shock wave imparts compressive stress on the calcified tissue. Shearing occurs on the lesion as the shock wave passes through the calcification. When the shock wave reaches the distal boundary of the calcification, the shock wave is both transmitted and reflected, inducing tensile stress that pulls the calcification apart. Further compressive stress is applied by the squeezing which occurs when the ultrasonic shock wave entering the calcium propagates faster than the remaining shock wave travelling outside the calcified region of tissue. These forces generated by IVL result in multi-plane and longitudinal fractures of the calcification in the tissue.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with conductive fluid (e.g., using a relatively low pressure of 2-4 atm) so that the balloon expands to contact the lesion but not to a degree that substantively displaces the lesion. Voltage pulses can then be applied across the electrodes of electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can include electrodes disposed within a closed volume other than an angioplasty balloon, such as a cap, balloons of variable compliancy, or other type of enclosure.

Critically, the calcified plaque remains in place following the shock waves; intimal calcium remains in the blood vessel lining and medical calcium remains in the muscle tissue surrounding the blood vessel. IVL generally does not cause the debulking or extirpation of tissue from a blood vessel wall. However, following the IVL shock wave, the hardened lesion is fractured and does not have the mechanical strength to resist against the expansion of a balloon. Thus, following delivery of IVL shock wave therapy, the catheter can be expanded or moved such that the modified underlying lesion can be moved or displaced along the blood vessel, similar to how a plain angioplasty balloon can treat non-calcified plaque. In some approaches, expansion of the balloon can be done in sequence with or concurrent with delivery of the IVL therapy.

Accordingly, the IVL process can also be considered different from standard atherectomy procedures and different from cutting or scoring balloons at least in that IVL cracks calcium but does not liberate the calcium from the tissue. Hence, generally speaking, IVL systems should not require aspiration nor embolic protection. Accordingly, IVL does not carry the same degree of risk of embolism, perforation, dissection, or other damage to vasculature as atherectomy procedures or angioplasty procedures using cutting or scoring balloons. In further contrast with cutting techniques, due to the compliance of a normal blood vessel and non-calcified plaque, the shock waves produced by IVL do not modify the normal healthy vessel tissue or non-calcified plaque. In other words, the shock waves from IVL do not have an adverse clinical impact on soft tissues while treating the hardened calcified anatomy.

For laser generation of acoustic shock waves, a laser pulse is transmitted into and energy from the laser is absorbed by a fluid within the catheter, optionally with a target to act as catalyst for the laser absorption. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of electrohydraulic and laser-based IVL devices are not intended to be a comprehensive list of potential energy sources to create the ultrasonic IVL shock waves.

For treating longer lesions, shock wave catheters may require longer balloons. However, maintaining concentricity or centering of shock wave emitters, which are typically mounted on an inner shaft of the catheter, within a longer balloon may be challenging, especially when treating lesions in curved vasculature. When emitters become off-center, they may get close to or in contact with the balloon wall, which may lead to pinhole creation when shock waves are generated, which in turn may lead to device failure.

According to an aspect, systems, devices, and methods for treating lesions of a body lumen utilize catheters that are configured to improve centering of one or more shock wave emitters within one or more enclosure(s) enclosing the one or more shock wave emitters. The improved centering enables catheters to have increased longitudinal distribution of shock wave emitters, enabling the treatment of longer lesions without requiring repositioning of the catheter. In some examples, a catheter includes an emitter centering feature in the form of one or more narrowing regions (also referred to herein as narrowings) that fluidly connect segments of the enclosure, each segment of the enclosure enclosing at least one shock wave emitter. In other examples, the catheter includes separate enclosures enclosing different sets of one or more shock wave emitters. In either case, the narrowings or separate enclosure may keep the inner shaft centered within the enclosure as the catheter is navigated through tortuous vasculature.

Existing shock wave catheters having long enclosures can have limited efficacy in tortuous vasculature due to the enclosure flexing inwardly toward the inner shaft supporting the shock wave emitters when the catheter is bent. If the emitters are in contact with the enclosure in this bent configuration and shock waves are generated at the emitters, the emitter(s) can rupture the enclosure, and/or the shock wave treatment applied to the lesion can exceed the intended parameters. Accordingly, using the shock wave catheters having an enclosure with narrowed regions (or multiple enclosures) disclosed herein, the emitters can remain centered in the enclosure(s), thus ensuring a safe operating distance between the shock wave emitters and walls of the enclosure, in turn preserving the integrity of the enclosure and adhering to prescribed operating conditions of the shock wave catheter.

The shock wave catheters disclosed herein can include at least one fluid lumen for filling and/or purging fluid (e.g., liquid, gas bubbles) from the enclosure(s) (or segments thereof). For example, a shock wave catheter disclosed herein may include a dedicated fluid filling lumen and a dedicated fluid purging lumen. The fluid lumens can include one or more openings corresponding to each of the respective enclosures or enclosure segments of a catheter. Additionally, or alternatively, shock wave catheters disclosed herein can include dedicated fluid filling and/or purging lumens for each of the respective enclosures or enclosure segments of a catheter.

The shock wave catheters disclosed herein can include one or more radiopaque markers arranged proximate to the emitter centering features for locating the shock wave catheter (e.g., in particular, the emitter centering feature of the catheter) during use.

As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement. Furthermore, numerical designators such as “first,” “second,” “third,” “fourth,” etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement. It should be further appreciated that any disclosure of a numerical range as a boundary term or inequality term is similarly inclusive of any numerical increment or gradation within the given range; e.g., recitation of a parameter that is “at least a defined value, where the defined value ranges from 5% to 50%” supports the disclosure of that parameter being “at least 5%”, “at least 50%”, “at least 37%”, “at least 42.4%”, and the like.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an electrode assembly. In the context of the present disclosure, the term “emitter” broadly refers to the region of an electrode assembly where the current transmits across the electrode pair, generating a shock wave. The term “emitter sheath” or “emitter band” (which are used interchangeably) refers to a sheath/band of conductive material that may form one or more electrodes of one or more electrode pairs, thereby forming a location of one or more emitters.

Components of emitters, including electrodes and emitter sheaths/bands, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

For treatment of an occlusion in a blood vessel, the voltage pulse applied by a power source, including any of the power sources described herein (which may also be referred to herein as voltage sources or pulse generators), is typically in the range of from about five hundred to three thousand volts (500 V-3,000 V). In some implementations, for the treatment of stenosis in a blood vessel or of another anatomical feature, the voltage pulse applied by the voltage source can be up to about fifteen thousand volts (15,000 V) or higher than fifteen thousand volts (15,000 V). The pulse width of the applied voltage pulses ranges between one microsecond and six microseconds (1-6 μs). The repetition rate or frequency of the applied voltage pulses may be between about 1 Hz and 10 Hz. The total number of pulses applied by the power source to a treatment device (e.g., an IVL catheter) can be, for example, sixty (60) pulses, eighty (80) pulses, one hundred twenty (120) pulses, three hundred (300) pulses, or up to five hundred (500) pulses, or any increments of pulses within this range. Further implementations of power sources can deliver greater than 500 pulses to a treatment device. Alternatively or additionally, in some examples the power source may be configured to deliver a packet of micro-pulses having a sub-frequency between about 10 Hz-10 kHz. The preferred voltage, repetition rate, and number of pulses for any given IVL device or treatment may vary depending on factors such as the size, length, eccentricity, nodularity, or orientation of the lesion, the extent of lesion or tissue calcification, the size of the blood vessel, the attributes of the patient (e.g., age, gender, predisposition to cardiac disease, etc.), or the stage of treatment. In delivering a treatment regime, a physician may start with low energy shock waves and increase the energy as needed during the procedure, or vice versa. The amount of power delivered for shock waves may further vary during the course of a procedure, following a predetermined sequence of energy increases or decreases, or by changing the amount of energy delivered in response to sensor data obtained prior to and/or during the IVL treatment procedure. The magnitude of the shock waves can be controlled by controlling the voltage, current, duration, and repetition rate of the pulsed voltage from the power source.

In some embodiments, an IVL catheter may be an “over-the-wire-type” (OTW) catheter. In an OTW catheter, the guidewire entry point to the catheter is at the distal end of the catheter tip, extends through the full length of the catheter through a lumen (alternatively referred to as a “guidewire lumen” and sometimes is structurally also a “central lumen”), and exits from the proximal end of the catheter via a guidewire port. In other embodiments, an IVL catheter is a “rapid exchange” (Rx) catheter. An Rx design allows just a portion of the catheter to ride over a guidewire. The guidewire entry point to the catheter is at the distal end of the catheter tip, and the extends along a pathway (e.g., a dedicated guidewire lumen) through a portion of the catheter until it reaches an exit port on the side of the catheter. The guidewire exit port is generally located at a position behind (proximal from) the distal tip of the catheter, and moreover the exit port is located along the length of a catheter proximal to a functional region of the catheter (e.g., an expandable member or a tensioned member having shock wave emitters). The location of an Rx catheter guidewire exit port can vary for a given design, generally being positioned from fifteen to thirty-five centimeters (15-35 cm) behind the distal tip, although it is appreciated that in some implementations the exit port can be less than 15 cm or greater than 35 cm behind the distal tip of the catheter. Thus in use, a guidewire is delivered into the anatomy of a patient, the proximal end of the guidewire (outside the patient) is fed into the distal end opening of the catheter, and the catheter is run along the guidewire until it reaches the target tissue at the distal end of the guidewire (inside the patient); the effective difference between an OTW and an Rx catheter is where the guidewire exits the catheter. The selection between an OTW design and an Rx design is driven by factors including (but not limited to): anatomy to be treated (e.g., coronary vasculature vs. peripheral vasculature); the length of guidewire to be used; the trackability, stiffness, torque transmission, and deliverability of the catheter; the profile and cross-section of the catheter, the ability to exchange a wire when the catheter is past a stenosis; positioning of the distal end of a catheter close to the end of a guidewire and further obtaining positional confirmation of the catheter.

As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, and 10,709,462, in U.S. Publication No. 2021/0085383, and in U.S. patent application Ser. No. 18/586,299, all of which are incorporated herein by reference in their entireties. Other catheter designs have improved the delivery of shock waves, for instance, by specific electrode construction and configuration thereby directing shock waves in a forward direction to break up tighter and harder-to-cross occlusions in vasculature. Examples of forward-biased or firing-firing catheter designs can be found in U.S. Pat. Nos. 10,966,737, 11,478,261, and 11,596,423, in U.S. Publication Nos. 2023/0107690 and 2023/0165598, and in U.S. patent application Ser. No. 18/524,575 and Ser. No. 18/680,853, all of which are incorporated herein by reference in their entireties.

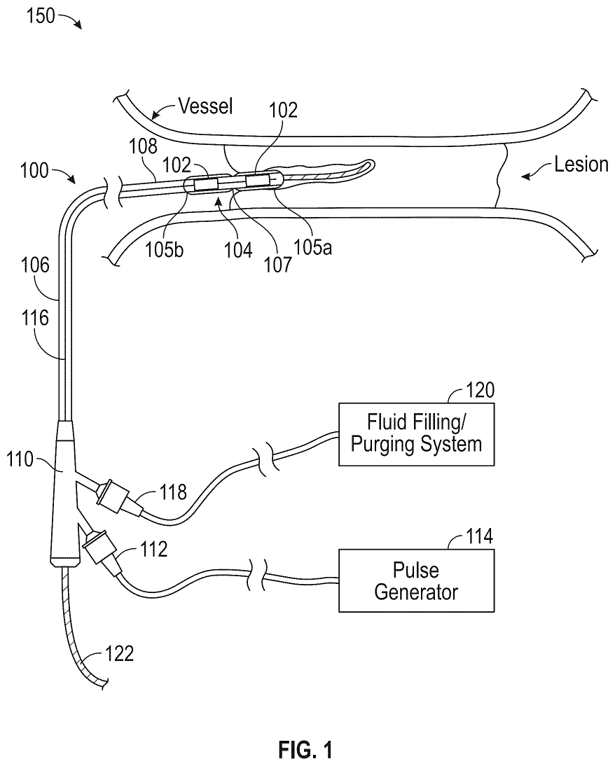

FIG. 1 illustrates an exemplary shock wave catheter system 150 that includes a shock wave catheter 100 configured for treating a lesion of a body lumen (e.g., a vessel). In some examples, the catheter 100 is configured for treating peripheral (e.g., below-the-knee) vasculature. The shock wave catheter 100 includes one or more shock wave emitters 102 disposed at a distal portion of the catheter 100 for generating shock waves that can treat the lesion.

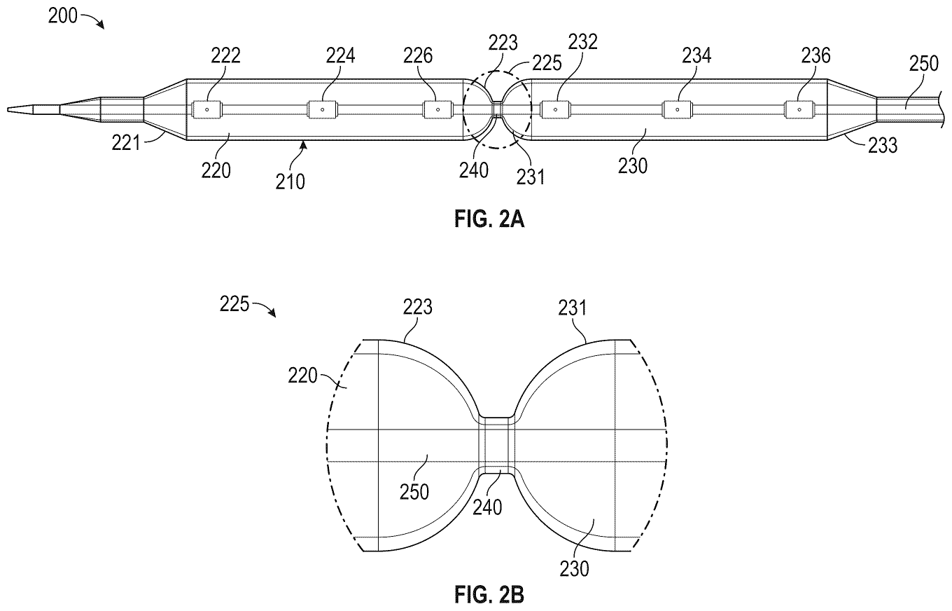

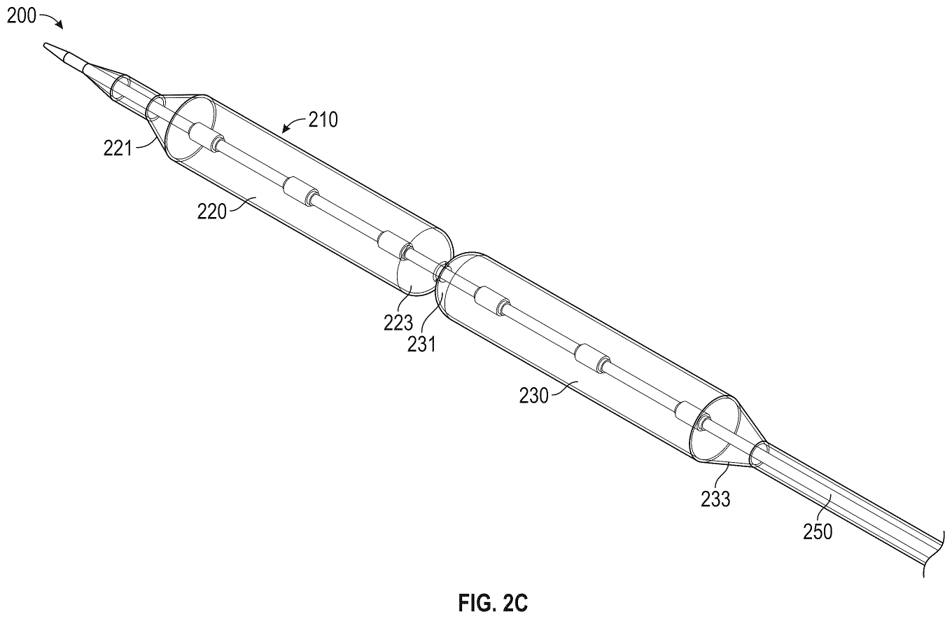

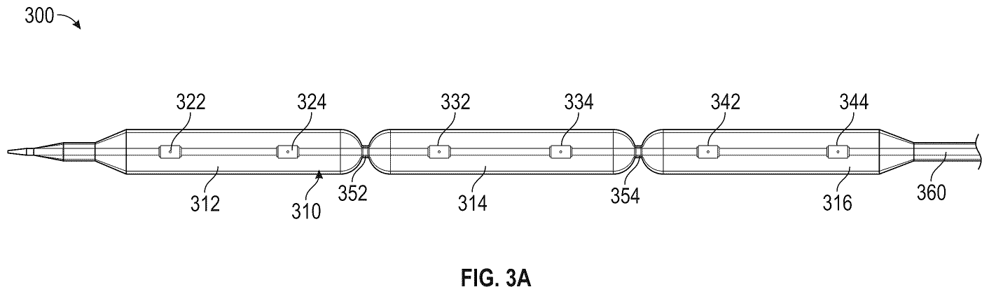

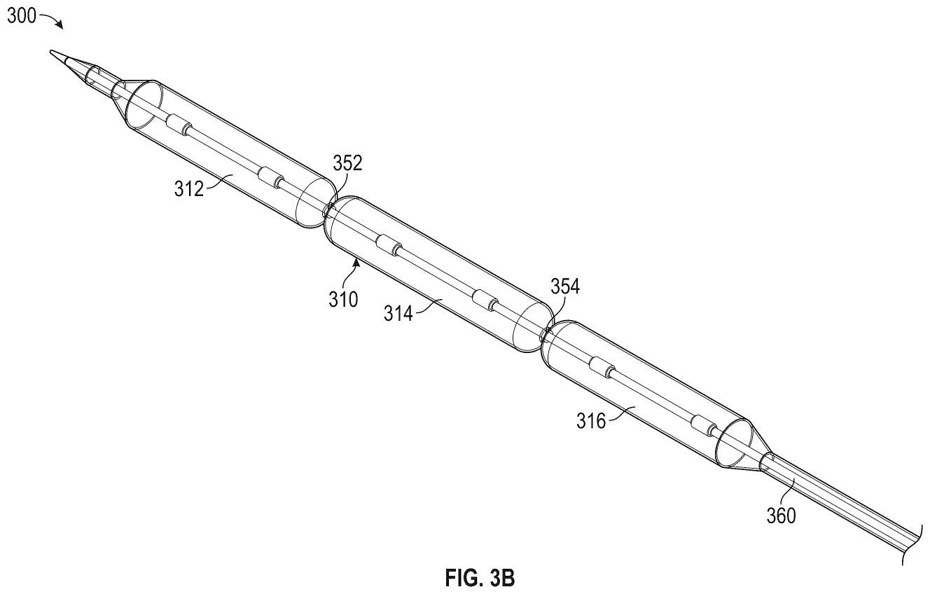

The catheter 100 includes one or more enclosures 104 disposed at the distal portion of the catheter 100. The enclosure 104 can enclose, or surround, the one or more shock wave emitters 102. In this way, the enclosure 104 can be filled with a conductive fluid (e.g., water, saline) that facilitates shock wave generation at the shock wave emitters 102. As described herein in greater detail with respect to at least FIGS. 2A-2C and 3A-3B, the enclosure 104 can include one or more segments, such as a distal segment 105a and a proximal segment 105b. The enclosure 104 can include an emitter centering feature, such as a narrowing 107, that keeps the shock wave emitters 102 centered within the one or more enclosures 104. Described in greater detail below, the narrowing 107 can fluidly connect the distal segment 105a and proximal segment 105b.

The catheter 100 includes an elongate tube 106 configured to be at least partially inserted in the vessel of a subject. The elongate tube 106 can include an inner shaft portion 108 that extends at least partially through the one or more enclosures 104. The enclosures 104 can be sealed to the elongate tube 106. The inner shaft portion 108 can support the shock wave emitter(s) 102. For example, the shock wave emitter(s) 102 can be mounted on the inner shaft portion 108. Additionally, or alternatively, the shock wave emitters 102 can be arranged within the inner shaft portion 108.

The shock wave catheter 100 can include a handle 110 that remains outside of the subject during a procedure. The handle 110 may include at least one port 112 that facilitates connection (e.g., a wired connection) between the one or more shock wave emitters 102 and a pulse generator 114. The pulse generator 114 can be a high-voltage source or a laser source. The energy (e.g., voltage) pulses generated by the pulse generator 114 can cause formation of shock waves at the one or more shock wave emitters 102. For example, the pulse generator 114 may generate voltage pulses with a frequency between 1 Hz and 5 Hz. The pulse generator 114 may generate voltage pulses with an amplitude between 0.5 kV and 15 kV.

The shock wave catheter 100 may include one or more fluid lumens 116 for delivering fluid (e.g., conductive fluid) to the one or more enclosures 104. In some examples, the one or more fluid lumens 116 can be configured for removing fluid (e.g., liquid, gas bubbles) from the one or more enclosures 104. In some examples, one or more of the fluid lumens 116 can be configured for delivering fluid to the body lumen. For example, the one or more fluid lumens 116 may include a fluid lumen extending through or along the elongate tube 106 that can be used for flowing an irrigation solution (e.g., saline or contrast) into the body lumen to clear blood or other bodily fluid.

The handle 110 may include one or more fluid ports 118 coupled to the one or more fluid lumens 116 for delivering fluid to and/or removing fluid from the shock wave catheter 100 (i.e., the one or more enclosures 104 of the catheter 100). The shock wave catheter system 150 can include a fluid filling and/or purging system 120 that facilitates the fluid delivery to and/or removal from the catheter 100. For example, the fluid filling/purging system 120 can remove gas bubbles from the enclosures 104 that may form during shock wave generation. In some examples, the fluid filling/purging system 120 is configured to apply a vacuum pressure to the enclosures 104 to remove fluid from the enclosures 104.

The shock wave catheter 100 can be advanced within the body lumen (e.g., vessel) such that the one or more enclosures 104 of the catheter 100 are positioned proximate to the lesion. The shock wave catheter 100 may generate shock waves by first filling the enclosure 104 with a conductive fluid (e.g., water or saline). When one or more suitable energy (e.g., voltage) pulses are applied to the shock wave emitters 102, an electrical arc can be formed in the conductive fluid within the enclosure 104. The formation of the electrical arc can create a shock wave that propagates outwardly toward the enclosure 104 for treating the lesion.

Shock wave emitters 102 may be uniformly spaced along the inner shaft portion 108 such that sonic output is generally uniform along a therapy delivering region of the catheter 100 (a region of the catheter extending in a longitudinal direction that can deliver shock wave treatment to a lesion). Shock wave emitters 102 may be electrically arranged in series, in parallel, in a combination of series and parallel, in multiple channels that can be independently controlled, or any combination of these arrangements.

The shock wave catheter 100 may include a guidewire 122 for navigating the shock wave catheter 100 through the body lumen and to the lesion. The guidewire 122 may be introduced to a corresponding lumen of the shock wave catheter 100 at the handle 110 and may extend through the elongate tube 106 and the enclosure(s) 104 prior to exiting the shock wave catheter 100 at the distal end of the elongate tube 106.

As described herein, the enclosure 104 of the shock wave catheter 100 can include one or more segments separated by one or more narrowing regions (or narrowings). FIGS. 2A-2C illustrate a distal region of shock wave catheter 200 including an enclosure 210 that has a distal segment 220 and a proximal segment 230, according to aspects of the disclosure. The catheter 200 can be used for catheter 100 in system 150. Catheter 200 may include one or more distal shock wave emitters 222, 224, and 226 enclosed within distal segment 220. Catheter 200 may include one or more proximal shock wave emitters 232, 234, and 236 enclosed within proximal segment 230. Distal segment 220 and proximal segment 230 may be separated by a narrowing region 240. The narrowing region 240 is illustrated in detail 225 shown in FIG. 2B. The narrowing region 240 can fluidly connect the distal segment 220 and proximal segment 230.

Catheter 200 includes an inner shaft 250, on which the emitters 222, 224, 226, 232, 234, 236 are mounted. By including narrowing region 240 in enclosure 210, the inner shaft 250 (and thus the emitters) can remain centered within the enclosure 210. In some embodiments, the enclosure does not contact the emitters 222, 224, 226, 232, 234, 236 when the catheter 200 (i.e., the inner shaft 250) is at a maximum curvature. The maximum curvature of the catheter 200 may refer to a maximum curvature that the catheter 200 is intended to have during normal use. In some examples, the maximum curvature of the catheter 200 is defined by the maximum possible elastic deformation of the inner shaft 250 of the catheter 200. As described herein, inner shaft 250 may be a portion of an elongate tube that extends to a proximal region of the catheter and includes one or more fluid lumens for delivering fluid (e.g., saline, fluoroscopic contrast agent, a mixture of saline and contrast agent) to fill enclosure 210. In various embodiments, enclosure 210 may be an angioplasty balloon.

The enclosure 210 may be composed of a medium-high durometer (e.g., having a Shore value between about 55-75 D) biocompatible material. Exemplary materials include, but are not limited to nylon, polyethylene terephthalate (PET), polyamide block copolymers, polyether block amide (PEBA) blends (e.g., PEBAX®), polyurethane blends, or combinations thereof. The material of the balloon can be uniform throughout. For example, the material of the distal and proximal segments 220, 230 can be the same as the narrowing region 240.

In some embodiments, distal segment 220 includes a distal cone 221 at its distal end and a rounded proximal end 223. In some embodiments, proximal segment 230 includes a proximal cone 233 at its proximal end and a rounded distal end 231. The opposite arrangement is also possible where the distal end of the distal segment 220 is rounded and/or the proximal end of the distal segment is conical. In embodiments, the proximal end of the proximal segment 230 is rounded and/or the distal end of the proximal segment 230 is conical. Having cones 221, 233 at distal and proximal ends of enclosure 210 may improve the catheter's deliverability and retractability. Other shapes of the segments of the enclosure 210 are also possible. For example, both ends of a given segment may be conical. Alternatively, both ends of a segment may be rounded.

FIG. 2B shows an enlarged view of narrowing region 240, according to aspects of the disclosure. As shown, narrowing region 240 is concentric with distal segment 220 and proximal segment 230 and thus keeps inner shaft 250 (and the emitters mounted thereon) concentric with enclosure 210. In some examples, a clearance between the narrowing region 240 and the inner shaft 250 can be between about 1-5 mm, such as between about 1-3 mm or about 2-4 mm.

According to aspects of the disclosure, enclosure 210 has a working length between 5 mm-120 mm, such as between 10 mm-100 mm, 50 mm-100 mm, or between 5 mm-10 mm. In some examples, enclosure 210 has a working length of no more than 5 mm. In some embodiments, enclosure 210 has a working length of 100 mm, 75 mm, 50 mm, 25 mm, 15 mm, 10 mm, 8 mm, 6 mm, 5 mm, or less. In some embodiments, enclosure 210 has a working length of greater than or equal to 2 mm, 4 mm, 5 mm, 6 mm, 8 mm, 10 mm, 15 mm, 25 mm, 50 mm, or 75 mm. In some embodiments, distal segment 220 and/or proximal segment 230 each has a working length between 4 mm-12 mm, such as between 6 mm-10 mm. In some examples, distal segment 220 and/or proximal segment 230 each has a working length less than 12 mm, such as less than 10 mm, 8 mm, 6 mm, 5 mm, 4 mm, 3 mm, 2 mm, or less. In some embodiments, distal segment 220 and/or proximal segment 230 has a working length greater than or equal to 1 mm, 2 mm, 3 mm, 4 mm, 5 mm, 6 mm, 8 mm, or 10 mm.

In some examples, the narrowing region 240 has a narrower width than the distal segment 220 and the proximal segment 230 when the enclosure 210 is filled and pressurized to between about two and four atmospheres. In some examples, the narrowing region 240 has a narrower width than the distal segment 220 and the proximal segment 230 when the enclosure 210 is filled and pressurized to less than 10 atm, 8 atm, 6 atm, 5 atm, 4 atm, 3 atm, or 2 atm. In some examples, the narrowing region 240 has a narrower width than the distal segment 220 and the proximal segment 230 when the enclosure 210 is filled and pressurized to greater than 0.5 atm, 1 atm, 2 atm, 3 atm, 4 atm, 5 atm, 6 atm, or 8 atm.

In some examples, a width of the narrowing region 240 is less than 50% of a width of at least one of the distal segment 220 and the proximal segment 230. In some examples, the width of the narrowing region 240 is less than 40%, 30%, 25%, 20%, or 15% of the width of the distal segment 220 and/or the proximal segment 230. In some examples, the width of the narrowing region 240 is greater than 5%, 10%, 15%, 20%, 25%, 30%, or 40% of the width of the distal segment 220 and/or the proximal segment 230.

In some examples, a length of the narrowing region 240 on the enclosure 210 is less than a distance between the distal-most shock wave emitter 232 in the proximal segment 230, and the proximal-most shock wave emitter 226 in the distal segment 220. For example, if the distance between the shock wave emitter 232 and the shock wave emitter 226 is about 1 mm, the length of the narrowing region 240 can be less than 1 mm. In this way, the narrowing region 240 avoids contact with the shock wave emitters positioned along the inner shaft 250. In some examples, the narrowing region 240 has a length between 0.1 mm-6 mm, such as between 0.1 mm-4 mm, or between 0.1 mm-2 mm. In some examples, the narrowing region 240 has a length of no more than 6 mm, 4 mm, 2 mm, 1 mm, or 0.5 mm. In some examples, the narrowing region 240 has a length of at least 0.1 mm, 0.5 mm, 1 mm, 2 mm, or 4 mm. The narrowing region may have many different geometries. For example, one lateral side of the narrowing region may be longer than the other lateral side of the narrowing region, e.g., for a curved enclosure.

In some embodiments, an enclosure of a shock wave catheter can include more than two segments respectively separated by narrowing regions. FIGS. 3A and 3B illustrate a distal region (i.e., the therapy delivering region) of a shock wave catheter 300 including an enclosure 310 having three segments—a distal segment 312, a middle segment 314, and a proximal segment 316. The catheter 300 can be used for catheter 100 in shock wave system 150. Shock wave catheter 300 includes a first narrowing region 352 fluidly connecting segments 312, 314 and a second narrowing region 354 fluidly connecting segments 314, 316. Unless explicitly stated otherwise herein, any features of the catheter 200 described with respect to FIGS. 2A-2C can be applicable to catheter 300.

The shock wave catheter 300 may include one or more distal shock wave emitters 322, 324 contained within distal segment 312 of enclosure 310. The catheter 300 may include one or more middle shock wave emitters 332, 334 contained within middle segment 314 of enclosure 310. The catheter 300 may include one or more proximal shock wave emitters 342, 344 contained within proximal segment 316 of enclosure 310. Shock wave emitters 322, 324, 332, 334, 342, 344 may be positioned within and/or along an inner shaft 360 of the shock wave catheter 300.

In some embodiments, enclosure 310 has a working length greater than 10 mm, such as 12 mm, 14 mm, 16 mm, 18 mm, 20 mm, or more. In some embodiments, enclosure 310 has a working length less than or equal 20 mm, 18 mm, 16 mm, 14 mm, or 12 mm. In some embodiments, enclosure 310 has a working length of about 12 mm. In some embodiments, each segment of enclosure 310 has a length less than 5 mm, such as less than 4 mm, less than 3 mm, or less than 2 mm. In some examples, the segments of enclosure 310 have a length greater than 1 mm, such as 2 mm, 3 mm, or 4 mm. The lengths of the various segments can be the same as one another or can vary amongst the segments.

While enclosure 210 is shown with two segments and enclosure 310 is shown with three segments, other longer enclosures may include more than three segments, each segment having at least one shock wave emitter. For example, an exemplary shock wave emitter may include two, three, four, five, six, or more enclosure segments, each enclosure segment surrounding one, two, three, four, or more shock wave emitters. In some examples, a catheter may include n-1 narrowing regions for every n enclosure segments. In some examples, a catheter may include a narrowing region every 4-12 mm along a length of the enclosure, such as every 6 mm, 8 mm, or 10 mm. For example, a 120 mm enclosure may include anywhere between 10 and 30 narrowing regions separating several enclosure segments. One or more of the enclosure segments may be the same length, or the enclosure segments may vary in length across the enclosure.

Including additional segments to the enclosure allows the therapy delivering region of the catheter to become longer while also ensuring shock wave emitters do not touch and/or get too close to the enclosure wall. In embodiments where the catheter includes a single enclosure having multiple segments, the inner shaft of the catheter may only be adhered to the enclosure at its distal and proximal ends and not adhered at the narrowing regions. This design may allow a single fluid lumen to fill and/or inflate the multiple segments of the enclosure.

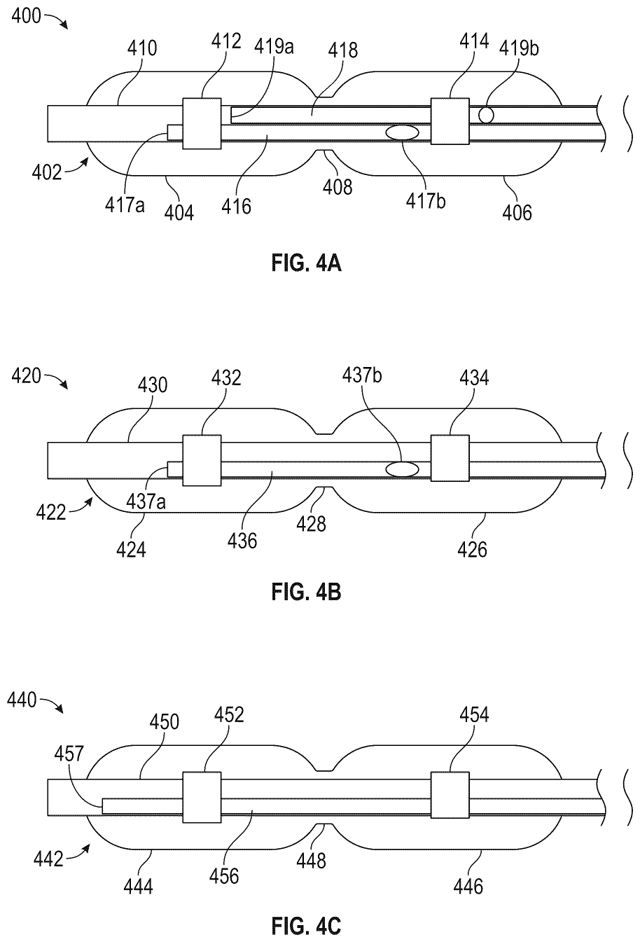

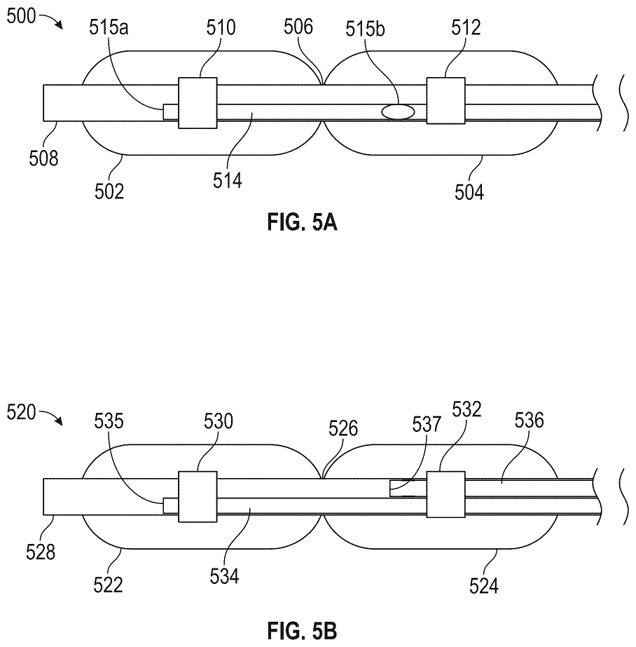

Inner shafts 250, 360 may include one or more lumina for delivering fluid to the segments of the enclosure. Inner shafts 250, 360 may additionally or alternatively include lumina and/or grooves for receiving energy guides (e.g., electrical wires) that connect the shock wave emitters of the catheter to an energy source (e.g., a voltage pulse generator, such as pulse generator 114 of system 150). FIGS. 4A-4C and 5A-5B illustrate exemplary shock wave catheters having various arrangements of fluid lumens for filling and/or removing fluid from enclosures. The shock wave catheters illustrated in FIGS. 4A-4C and 5A-5B may be representative of catheters 200, 300 and thus can be used for catheter 100 in system 150.

FIG. 4A illustrates a distal region of a shock wave catheter 400 including an enclosure 402 having a distal segment 404 and a proximal segment 406 that are fluidly connected at a narrowing 408 of the enclosure 402. The catheter 400 may include an inner shaft 410 that extends through the enclosure 402. The catheter 400 may include one or more shock wave emitters 412 located within the distal segment 404 and one or more shock wave emitters 414 located within the proximal segment 406, the shock wave emitters 412, 414 supported by the inner shaft 410.

The catheter 400 may include a first fluid lumen 416 for filling the enclosure 402 and a second fluid lumen 418 for evacuating, or purging, fluid (e.g., liquid and/or gas bubbles) from the enclosure 402. The first fluid lumen 416 and/or the second fluid lumen 418 can be lumens of the inner shaft 410. In some examples, the first fluid lumen 416 and/or the second fluid lumen 418 are lumens of one or more elongate tubes extending coaxial with and/or alongside the inner shaft 410, within the enclosure 402.

The first fluid lumen 416 can include one or more openings 417a, 417b for filling the different segments of the enclosure 402. For example, the first fluid lumen 416 can include a distal opening 417a for filling the distal segment 404 of the enclosure 402, and a proximal opening 417b for filling the proximal segment 406 of the enclosure 402. The second fluid lumen 418 can include one or more openings 419a, 419b for purging fluid (e.g., liquid and/or gas bubbes) from the different segments of the enclosure 402. For example, the second fluid lumen 418 can include a distal opening 419a for purging the distal segment 404 of the enclosure 402, and a proximal opening 419b for purging the proximal segment 406 of the enclosure 402.

The openings of the fluid lumens 416, 418 can be arranged anywhere within the respective enclosure segments. In some examples, the openings 419a, 419b for purging gas bubbles from the enclosure segments can be disposed proximate to the shock wave emitters 412, 414 to efficiently evacuate any gas bubbles generated from shock wave formation at the emitters. In the instance a given enclosure segment encloses a plurality of shock wave emitters, the fluid lumen 418 may include a corresponding opening for each of the shock wave emitters.

It is to be understood that a catheter enclosure can include any number of segments, and, in some examples, can include fluid filling and/or fluid purging openings corresponding to one or more of the segments. For example, the catheter may include at least one of a fluid filling opening and a fluid purging opening in each of the respective enclosure segments. In some examples, the catheter includes a fluid filling opening and/or a fluid purging opening in every other, every two, every three, etc. enclosure segments. In some examples, the catheter includes a single fluid filling opening, and a plurality of fluid purging openings. In some examples, the catheter includes a plurality of fluid filling openings and a single fluid purging opening.

FIG. 4B illustrates a distal region of a shock wave catheter 420 including an enclosure 422 that, like catheter 400, has a distal segment 424 and a proximal segment 426 that are fluidly connected at a narrowing 428 of the enclosure 422. Similar to catheter 400 described with respect to FIG. 4A, the catheter 420 may include an inner shaft 430 that extends through the enclosure 422. The catheter 420 may include one or more shock wave emitters 432 located within the distal segment 424 and one or more shock wave emitters 434 located within the proximal segment 426, the shock wave emitters 432, 434 supported by the inner shaft 430.

Catheter 420 may differ from catheter 400 in that catheter 420 may include a single fluid lumen 436 for filling the enclosure 422 and/or evacuating fluid (e.g., liquid, gas bubbles) from the enclosure 422. The fluid lumen 436 can be a lumen of the inner shaft 430 or can be of a separate elongate tube that extends alongside or coaxial with the inner shaft 430. The fluid lumen 436 can include one or more openings 437a, 437b for filling and/or purging fluid from the enclosure segments. For example, the fluid lumen 436 can include a distal opening 437a for filling and/or purging fluid from the distal segment 424 of the enclosure 422, and a proximal opening 437b for filling and/or purging fluid from the proximal segment 426 of the enclosure 422.

FIG. 4C illustrates a distal region of a shock wave catheter 440 including an enclosure 442 that, like catheters 400, 420 has a distal segment 444 and a proximal segment 446 that are fluidly connected at a narrowing 448 of the enclosure 442. Similar to catheters 400, 420 described with respect to FIGS. 4A and 4B, respectively, the catheter 440 may include an inner shaft 450 that extends through the enclosure 442. The catheter 440 may include one or more shock wave emitters 452 located within the distal segment 444 and one or more shock wave emitters 454 located within the proximal segment 446, the shock wave emitters 452, 454 supported by the inner shaft 450.

Catheter 440 may differ from catheters 400, 420 in that catheter 440 may include a single fluid lumen 456 having a single opening 457 for filling and/or purging fluid from all the enclosure segments. The opening 457 can be disposed at the distal portion of the catheter 440, i.e., within the distal segment 444 of the enclosure 442. In this way, the segments of the enclosure 442 can be sequentially filled from distal-most to proximal-most enclosure segment. In some examples, gas bubbles generated from shock wave formation at the emitters 452, 454 may migrate to the distal portion of the enclosure 442. Thus, positioning the opening 457 of the fluid lumen 456 at the distal portion of the enclosure 442 can efficiently capture the gas bubbles once migrated. Alternatively, the opening 457 of the fluid lumen 456 can be positioned at a proximal portion of the enclosure 442.

In some embodiments, instead of a single enclosure having multiple segments (e.g., a distal segment and a proximal segment), a shock wave catheter may include separate distal and proximal enclosures. FIGS. 5A-5B illustrate diagrams of exemplary shock wave catheters 500, 520 having separate distal and proximal enclosures that can be used for catheter 100 in system 150. In such embodiments, each enclosure may be bonded, or sealed, to the inner shaft at distal and proximal ends of each enclosure. Each enclosure may be in fluid communication with a fluid lumen of the inner shaft such that the distal and proximal enclosures are filled by a fluid together. In some embodiments, the distal and proximal enclosures may be separately connected to a fluid source by separate lumina that extend within the inner shaft. Thus, the distal and proximal enclosures can be filled separately.

FIG. 5A illustrates a distal region of a shock wave catheter 500 including a distal enclosure 502 and a proximal enclosure 504. The catheter 500 can include an emitter centering feature, such as a constriction 506 disposed between the distal enclosure 502 and the proximal enclosure 504 that fluidly isolates the enclosures from one another. The catheter 500 may include an inner shaft 508 that extends through the enclosures 502, 504. The catheter 500 may include one or more shock wave emitters 510 located within the distal enclosure 502 and one or more shock wave emitters 512 located within the proximal enclosure 504, the shock wave emitters 510, 512 supported by the inner shaft 508.

The catheter 500 can include one or more fluid lumens 514 for filling and/or evacuating fluid from the enclosures 502, 504. For example, the catheter 500 can include a single fluid lumen 514 in fluid communication with each of enclosures 502, 504 for both filling and evacuating fluid from both of the enclosures 502, 504. In some examples (e.g., as described above with respect to FIG. 4A), the catheter 500 can include separate fluid lumens for filling and evacuating fluid from the enclosures, respectively. The fluid lumen 514 can be a lumen of the inner shaft 508, or can be a lumen of a separate elongate tube that is coaxial with or runs alongside the inner shaft 508 through the enclosures 502, 504. The fluid lumen 514 can include one or more openings 515a, 515b for filling and/or evacuating the fluid from the enclosures. For example, the fluid lumen 514 can include a distal opening 515a fluidly connected with the distal enclosure 502 for filling and/or evacuating fluid from the distal enclosure 502. The fluid lumen 514 can include a proximal opening 515b fluidly connected with the proximal enclosure 504 for filling and/or evacuating fluid from the proximal enclosure 504. As described herein, in some examples, the openings 515a, 515b may be arranged proximate to the shock wave emitters 510, 512 for removing gas bubbles as they are generated during shock wave formation.

FIG. 5B illustrates a distal region of a shock wave catheter 520 including a distal enclosure 522 and a proximal enclosure 524. Like catheter 500, the catheter 520 can include an emitter centering feature, such as a constriction 526 disposed between the distal enclosure 522 and the proximal enclosure 524 that fluidly isolates the enclosures from one another. The catheter 520 may include an inner shaft 528 that extends through the enclosures 522, 524. The catheter 520 may include one or more shock wave emitters 530 located within the distal enclosure 522 and one or more shock wave emitters 532 located within the proximal enclosure 524, the shock wave emitters 530, 532 supported by the inner shaft 528.

The catheter 520 may differ from catheter 500 described with respect to FIG. 5A in that the catheter 520 can include a plurality of fluid lumens 534, 536 for respectively filling and/or purging fluid from the enclosures. For example, the catheter 520 can include a first fluid lumen 534 in fluid communication with the distal enclosure 522 for filling and/or purging fluid from the distal enclosure 522, and a second fluid lumen 536 in fluid communication with the proximal enclosure 524 for filling and/or purging fluid from the proximal enclosure 524. One or more of the fluid lumens 534, 536 can be lumens of the inner shaft 528. In some examples, one or more of the fluid lumens 534, 536 are lumens of a separate elongate tube that is coaxial with and/or runs alongside the inner shaft 528.

The fluid lumen 534 can include one or more openings 535 for filling and/or purging fluid from the distal enclosure 522. For example, the fluid lumen 534 can include a single opening 535 for both filling and evacuating fluid from the distal enclosure 522. In some examples (e.g., as described with respect to FIGS. 4A and 4B), the fluid lumen 534 can include separate openings for filling and evacuating fluid from the distal enclosure 522, respectively.

The fluid lumen 536 can include one or more openings 537 for filling and/or purging fluid from the proximal enclosure 524. For example, the fluid lumen 536 can include a single opening 537 for both filling and evacuating fluid from the proximal enclosure 524. In some examples, the fluid lumen 536 can include separate openings for filling and evacuating fluid from the proximal enclosure 524, respectively.

An exemplary shock wave catheter having a plurality of enclosures can include more than two enclosures, such as three, four, five, six, or more enclosures respectively separated by constrictions. In some examples, such a catheter can include one or more fluid lumens for filling and/or purging fluid from the enclosures, the fluid lumens including corresponding openings for each of the enclosures. For example, a given fluid lumen may include two, three, four, five, six, or more openings, each opening corresponding to a given enclosure. In some examples, a fluid lumen of an exemplary shock wave catheter includes more than one opening corresponding to each enclosure, such as two, three, four or more openings for a given enclosure. The openings can be configured for filling and/or removing fluid from the enclosure.

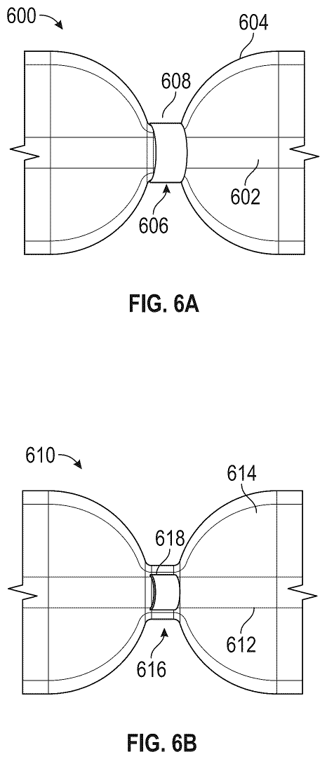

In some examples, the shock wave catheters disclosed herein can include one or more radiopaque markers for locating and positioning the shock wave catheter device in the patient during use. In particular, radiopaque markers may be used to locate one or more emitter centering features of the shock wave catheter. The shock wave catheter may be positioned in the vasculature based on the position of the emitter centering features (e.g., one or more narrowings) of the enclosure. FIGS. 6A and 6B illustrate portions of exemplary shock wave catheters 600, 610 having radiopaque markers arranged at an emitter centering feature of the catheter. The catheters 600, 610 can be representative of any of the catheters described herein and thus can be used for catheter 100 of system 150.

FIG. 6A illustrates a portion of a shock wave catheter 600 including an inner shaft 602 and an enclosure 604. The enclosure 604 can include a narrowing 606. The narrowing 606 of the enclosure 604 can include one or more radiopaque markers 608 for locating the catheter 600 using fluoroscopic imaging.

FIG. 6B illustrates a portion of a shock wave catheter 610 including an inner shaft 612 and an enclosure 614. The enclosure 614 can include a narrowing 616. The inner shaft 612 can include one or more radiopaque markers 618 for locating the catheter 610 using fluoroscopic imaging. The radiopaque markers 618 can be disposed on the inner shaft 612 such that the markers are proximate to the narrowing(s) 616 of the enclosure 614.

In some examples, a shock wave catheter can include a plurality of enclosure narrowings and a plurality of radiopaque markers, at least one of the radiopaque markers arranged proximate to a narrowing of the enclosure. In some examples, a shock wave catheter can include one or more constrictions (e.g., rather than narrowings). In this example, the shock wave catheter can include one or more radiopaque markers on the inner shaft and/or on a portion of an enclosure of the catheter, the radiopaque marker(s) arranged proximate to the constriction(s).

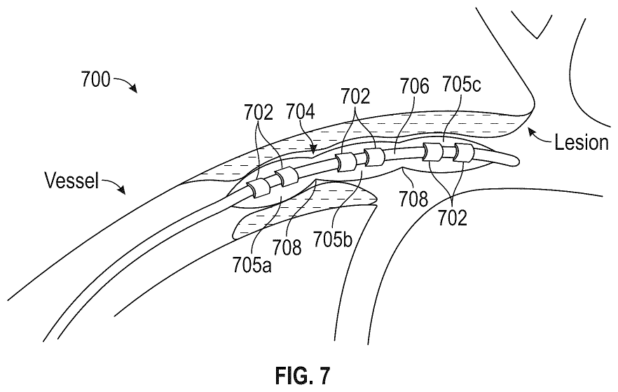

FIG. 7 illustrates a portion of a shock wave catheter 700 being advanced within tortuous vasculature having a long lesion therein. The shock wave catheter 700 can be used for catheter 100 in system 150. In some examples, the vasculature is a vessel in the peripheral vasculature. The catheter 700 can include a plurality of shock wave emitters 702 enclosed by segments of an enclosure 704 and supported by an inner shaft 706. The enclosure 704 can include a proximal segment 705a, a middle segment 705b, and a distal segment 705c, segments connected by narrowing regions 708.

As shown in FIG. 7, when the catheter 700 is advanced through tortuous vasculature, the inner shaft 706 can bend to correspond to the curvature of the vasculature. When the inner shaft 706 is bent, the enclosure 704 can become offset with respect to the inner shaft 706, bringing the emitters 702 closer to the wall of the enclosure 704. However, due to the narrowing regions 708, the emitters 702 can remain spaced from the wall of the enclosure 704. In some embodiments, spacing is maintained between the inner shaft 706 and the segments 705a, 705b, 705c of the enclosure 704 at a maximum curvature of the catheter 700. As used herein, the maximum curvature of the catheter 700 refers to a maximum curvature that the catheter 700 is intended to have during normal use. In some examples, the maximum curvature may be defined by the maximum possible elastic deformation of the inner shaft 706.

The shock wave catheters disclosed herein can enable treatment of longer lesions, in particular longer lesions in curved vasculature such as that illustrated in FIG. 7. As shown, using the catheter 700 having an extended distribution of shock wave emitters 702 at the distal portion of the catheter 700, the lesion in the curved vasculature can be treated at once, without having to reposition the catheter 700.

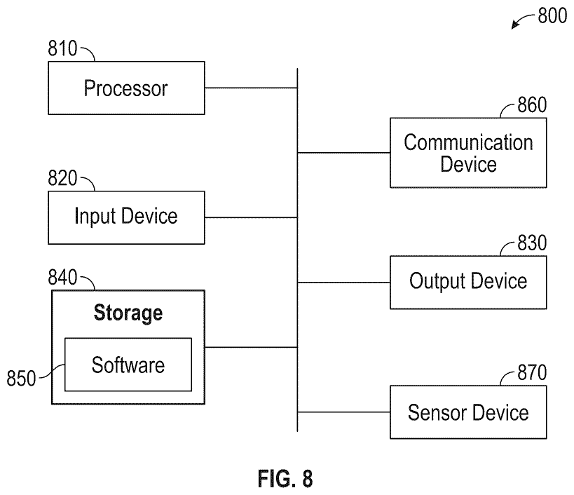

FIG. 8 illustrates an example of a computing system 800 configured to be used with any of the catheters or catheter systems described herein. System 800 can be a computer connected to a network, such as one or more networks of hospital, including a local area network within a room of a medical facility and a network linking different portions of the medical facility, or a wide-area network accessed through the internet or other means. System 800 can be a client or a server. System 800 can be any suitable type of processor-based system, such as a personal computer, workstation, server, handheld computing device (portable electronic device), such as a phone or tablet, or dedicated device. System 800 can include, for example, one or more of input device 820, output device 830, one or more processors 810, storage 840, and communication device 860. Input device 820 and output device 830 can generally correspond to those described above and can either be connectable or integrated with the computer.

Input device 820 can be any suitable device that provides input, such as a touch screen, keyboard or keypad, mouse, gesture recognition component of a virtual/augmented reality system, or voice-recognition device. Output device 830 can be or include any suitable device that provides output, such as a display, touch screen, haptics device, virtual/augmented reality display, or speaker.

Storage 840 can be any suitable device that provides storage, such as an electrical, magnetic, or optical memory including a RAM, cache, hard drive, removable storage disk, or other non-transitory computer-readable medium. Communication device 860 can include any suitable device capable of transmitting and receiving signals over a network, such as a network interface chip or device. The components of the computing system 800 can be connected in any suitable manner, such as via a physical bus or wirelessly.

Processor(s) 810 can be any suitable processor or combination of processors, including any of, or any combination of, a central processing unit (CPU), field programmable gate array (FPGA), and application-specific integrated circuit (ASIC). Software 850, which can be stored in storage 840 and executed by one or more processors 810, can include, for example, the programming that embodies the functionality or portions of the functionality of the present disclosure (e.g., as embodied in the devices/systems as described above).

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer-readable medium can include, but is not limited to, an electronic, magnetic, optical, electromagnetic, or infrared wired or wireless propagation medium.

System 800 may further include a sensor device 870 that provides sensor data for processing by processor 810. Sensor device 870, in some embodiments, may be an imaging sensor that provides imaging data, for a lesion being treated. In some embodiments, sensor device 870 may be a voltage sensor, a current sensor, a pressure sensor, a temperature sensor, an electromagnetic sensor, or an optical sensor for providing data about a state of the catheter or a lesion. Depending on the type of sensors used, sensor device 870 can be physically located on a catheter along a region that is configured to enter a patient or incorporated into a part of the catheter system that remains external to a patient during treatment. In some embodiments, sensor device 870 can include one or more of the sensor types identified here, in any combination. In some implementations, data collected by sensor device 870 may be displayed on output device 830, modified or corrected through input device 820, saved to storage 840, transmitted to a separate system via communication device 860, or any combination thereof.

System 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 800 can implement any operating system suitable for operating on the network. Software 850 can be written in any suitable programming language, such as C, C++, Java, or Python. In various examples, application software embodying the functionality of the present disclosure can be deployed in different configurations, such as in a client/server arrangement or through a Web browser as a Web-based application or Web service.

System 800 may be configured to selectively control the delivery of energy from one or more of energy sources (e.g., a voltage pulse generator or a light energy source) to one or more acoustic energy emitters (e.g., a forward-firing emitter, a radially-firing emitter, an unenclosed emitter, or an enclosed emitter) depending on input from input device 820.

System 800 may be configured to tune the energy properties of energy delivered to one or more of the above-described emitters based on tissue properties received from sensor device 870. Tissue properties may include lesion tissue type (e.g., calcific, thrombic, fibrotic), lesion morphology (e.g., thickness, length, eccentricity).

The elements and features of the example catheters and catheter systems illustrated throughout this specification and drawings may be rearranged, recombined, and modified without departing from the present disclosure. For instance, the number, placement, and spacing of shock wave generating regions or emitters can be modified and the number, placement, and spacing of the enclosures of catheters can be modified without departing from the present disclosure.

Although the catheter devices described herein have been discussed primarily in the context of treating coronary occlusions, such as lesions in vasculature, the catheter devices described herein can be used for a variety of occlusions, such as occlusions in the peripheral vasculature (e.g., above-the-knee, below-the-knee, iliac, carotid, etc.). For further examples, various embodiments may be used for treating soft tissues, such as cancer and tumors (i.e., non-thermal ablation methods), blood clots, fibroids, cysts, organs, scar and fibrotic tissue removal, or other tissue destruction and removal treatments. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal, and destruction of tissue, or used in place of thermal treatments or cauterization for venous insufficiency and fallopian ligation (i.e., for permanent female contraception).

In one or more examples, the electrode assemblies and, catheters described herein could also be used for tissue engineering methods, for instance, for mechanical tissue decellularization to create a bioactive scaffold in which new cells (e.g., exogenous and endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It will be understood that the foregoing is only illustrative, and that various modifications, alterations and combinations can be made by those skilled in the art without departing from the scope and spirit of the disclosure. Any of the variations of the various catheters disclosed herein can include features described by any other catheters or combination of catheters herein. Furthermore, any of the methods can be used with any of the catheters disclosed. Accordingly, it is not intended that the systems, catheters, and methods described herein be limited, except as by the appended claims.

1. A shock wave catheter comprising:
an enclosure comprising a distal segment, a proximal segment, and a narrowing region fluidly connecting the distal segment and the proximal segment;
an inner shaft extending through the distal segment and the proximal segment;
a distal shock wave emitter supported by the inner shaft and located within the distal segment; and
a proximal shock wave emitter mounted on the inner shaft and located within the proximal segment.

2. The catheter of claim 1, wherein the distal emitter comprises a plurality of distal emitters and the proximal emitter comprises a plurality of proximal emitters.

3. The catheter of claim 1, wherein the enclosure comprises a middle segment located between the distal segment and the proximal segment and a plurality of narrowing regions separating each of the distal segment, the proximal segment, and the middle segment, and wherein the catheter further comprises a middle shock wave emitter mounted on the inner shaft and located within the middle segment.

4. The catheter of claim 1, wherein the enclosure is configured such that spacing is maintained between the inner shaft and the distal and the proximal segments at a maximum curvature of the catheter.

5. The catheter of claim 1, wherein at least one of the distal segment and the proximal segment has a working length of no more than 5 mm.

6. The catheter of claim 1, wherein the narrowing has a narrower width than the distal segment and the proximal segment when the enclosure is filled and pressurized to between two and four atmospheres.

7. The catheter of claim 1, wherein a width of the narrowing is less than 50% of a width of at least one of the distal segment and the proximal segment.

8. The catheter of claim 1, wherein a length of the narrowing is less than a distance between the distal and proximal shock wave emitters.

9. The catheter of claim 1, comprising a radiopaque marker disposed at the narrowing.

10. The catheter of claim 1, comprising a fluid lumen configured to fill the proximal segment and the distal segment with conductive fluid and to remove gas from the proximal segment and the distal segment of the enclosure.

11. The catheter of claim 10, wherein the fluid lumen comprises a single opening disposed at a distal portion of the shock wave catheter.

12. The catheter of claim 10, wherein the fluid lumen comprises a first opening in the proximal segment and a second opening in the distal segment of the enclosure.

13. The catheter of claim 1, comprising a first fluid lumen configured to fill the proximal segment and the distal segment of the enclosure with conductive fluid, and a second fluid lumen configured to remove gas from the proximal segment and the distal segment of the enclosure.

14. A system, comprising:
the shock wave catheter of claim 1; and
a pulse generator electrically coupled to the proximal shock wave emitter and the distal shock wave emitter, the pulse generator configured to generate voltage pulses that cause shock wave formation at the proximal shock wave emitter and the distal shock wave emitter.

15. A method for treating a lesion in a body lumen, comprising:
advancing a shock wave catheter within the body lumen such that an enclosure of the shock wave catheter is positioned proximate to the lesion, the enclosure of the catheter comprising a proximal segment, a distal segment, and a narrowing region fluidly connecting the proximal segment and the distal segment; and
generating shock waves by a proximal shock wave emitter of the shock wave catheter that is located within the proximal segment and a distal shock wave emitter of the shock wave catheter that is located within the distal segment to treat the lesion.

16. The method of claim 15, wherein advancing the shock wave catheter within the body lumen comprises bending the shock wave catheter such that the proximal and distal segment are proximate to the lesion, and wherein, when the shock wave catheter is bent, the proximal and distal shock wave emitters are spaced from the respective proximal and distal segments due to the narrowing region.

17. A shock wave catheter comprising:
a distal enclosure;
a proximal enclosure positioned proximally of the distal enclosure;
an inner shaft extending through the distal enclosure and the proximal enclosure;
a distal shock wave emitter supported by the inner shaft and enclosed by the distal enclosure; and
a proximal shock wave emitter supported by the inner shaft and enclosed by the proximal enclosure.

18. The catheter of claim 17, comprising a first fluid lumen in fluid communication with the distal enclosure and a second fluid lumen in fluid communication with the proximal enclosure.

19. The catheter of claim 18, wherein the distal enclosure and the proximal enclosure are configured to be filled separately.

20. The catheter of claim 17, comprising a fluid lumen fluidly connected to the distal enclosure and the proximal enclosure, the fluid lumen comprising a first opening fluidly connected to the distal enclosure and a second opening fluidly connected to the proximal enclosure.
